(19) United States
(12) Patent Application Publication
(10) Pub. No.: US 2017/0231265 A1
(43) Pub. Date: Aug. 17, 2017

(54) Green Leaf

(71) Applicant: Matthew Svedahl, Prior Lake, MN (US)

(72) Inventor: Matthew Svedahl, Prior Lake, MN (US)

(21) Appl. No.: 15/041,951

(22) Filed: Feb. 11, 2016

(51) Int. Cl. A24B 15/16; A24F 23/02; A61Q 11/00; A61K 8/19; A61K 8/02; A24B 15/30; A61K 8/97

(52) U.S. Cl. CPC A24B 15/16; A24B 15/303; A24F 23/02; A61K 8/97; A61K 8/19; A61K 8/0204; A61Q 11/00; A61K 2800/87

(57) ABSTRACT

The present invention provides a natural composition that could be placed between cheek and gum, for providing a person with a habitual chewing satisfaction the cleaning of their enamel. The composition includes powdered or pulverized green tea leaves, turmeric powder, sodium bicarbonate, and natural flavoring agents.

### FIELD OF INVENTION

The present invention relates to a chewing composition and in particular to a natural chewing composition.

### BACKGROUND OF INVENTION

Chewing provides mental satisfaction to the smokers and tobacco chewers having a chew pouch tucked into the gums and slowly helps to quit smoking and tobacco chewing. Regular consumption of tobacco or smoking leads to discoloration of enamel and slowly harms the tooth structure. Prior art discloses variety of tooth whitening chewing gums, whitening strips and trays which usually contain abrasive agents. The abrasive agents cause irritation to the gums and on long use may damage the teeth. For example, a published U.S. patent application Ser. No. 11/084,468 entitled “Teeth whitening candy with tartar removal and breath freshening properties” discloses a teeth whitening candy that enhance the removal of the tartar and plaque, which accumulates after meals. The candy comprises citric acid (or any combination of organic acids), tannic acid, calcium dioxide (or any other peroxides), sodium bicarbonate, titanium white, Arabic gum (or any organic gum), sodium laurel phosphate (or any other surfactants), flavoring agents, sweeteners; capable of whitening the teeth, breath freshening and dissolving tartar and calculus when sucking. Another, U.S. Pat. No. 7,390,518 issued to Cadbury Adams Usa, Llc., discloses a stain removing chewing gum composition comprising a gum base and a stain-removing complex consisting essentially of a stain-removing agent within a cavity of a cyclodextrin compound.

Thus, a need could be appreciated for an improved composition that is safe in use and provides multiple benefits to the user. Moreover, the composition should be economical, thus having a commercial advantage. The present invention addresses the problem of providing a natural composition that provides habitual chewing satisfaction and removes stains from the enamel.

### SUMMARY OF THE INVENTION

The present invention provides a chewing composition comprising natural ingredients enclosed in a wrap or pouch that is adapted to be place between the cheek and the gums for slowly releasing the ingredients into the oral cavity providing various health benefits to the user such as cleaning of teeth, providing habitual tobacco chewing satisfaction etc.

Another object of the present invention is that the chewing composition provides cleaning of the enamel.

Still a further object of the present invention is that the chewing composition helps to quit smoking and tobacco chewing.

In another embodiment, the chewing composition comprises green tea leaves, natural flavor enhancers, natural enamel whitening/cleaning agents, and baking soda.

In addition to the various objects and advantages of the present invention described with some degree of specificity above it should be obvious that additional objects and advantages of the present invention will become more readily apparent to those persons who are skilled in the relevant art from the following more detailed description of the invention.

### BRIEF DESCRIPTION OF THE DRAWINGS

For further clarifying various aspects of some embodiments of the present invention, a more particular description of the invention will be rendered by reference to specific embodiments thereof, which are illustrated in the appended drawing. It is appreciated that the drawing depicts only illustrated embodiments of the invention and are therefore not to be considered limiting of its scope. The invention will be described and explained with additional specificity and details through the use of the accompanying drawing in which:

FIG. 1 shows the compositional rolled into a mass;

FIG. 2 shows the rolled mass of FIG. 1 placed on a wrapper;

FIG. 3 shows a container for containing rolled wrappers of FIG. 2; and

FIG. 4 is the top view of the containing showing the rolled wrappers of FIG. 2.

### DETAIL DESCRIPTION OF THE INVENTION

The present invention provides a chew composition that includes natural ingredients for use in the oral cavity of a user for providing habitual chewing satisfaction while at the same time provides cleaning of enamel. The chew composition includes the use of green tea leaves that help to quit tobacco chewing and smoking. Green tea includes polyphenolic compounds known as catechins that are known to have widespread biological activities, including the prevention of tooth decay and oral cancer. Use of green tea helps to remove bad breath as the catechins have antibacterial properties. The green tea could be used in grounded or pulverized form, which slowly releases the active ingredients, such as, catechins in the oral cavity.

The chew composition further includes natural flavoring agents such as wintergreen, mint, and cherry, which provides a refreshing feeling to the mouth and removes bad breath. The composition further includes natural whitening agents such as abrasives that provide cleaning of the enamel by removing stains and plaques, and further enhances healing of the tissue. Preferably, turmeric powder could be used as a natural abrasive agent that further has excellent healing properties. The user's habitual tobacco chewing or smoking leads to discoloration of teeth and damages the gum tissue. Thus, the present invention provides for both cleaning of the enamel and healing of the gum tissue. As stated above, use of green tea leaves as a substitute for tobacco helps to quit tobacco chewing and smoking. Furthermore, sodium bicarbonate also known as baking powder could be added to aid in cleaning of the user's enamel. In another embodiment, other natural ingredients such as spices and herbs could be added, for example, clove buds which are well known for providing relief in tooth pains and further adds flavor and aroma to the composition. Similarly, coffee leaves could be added in addition to turmeric powder for providing cleaning action and flavor.

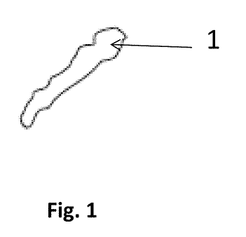

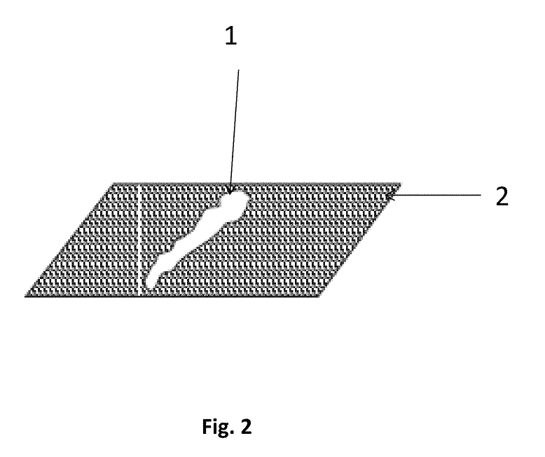

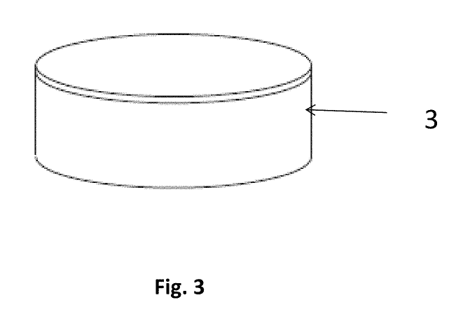

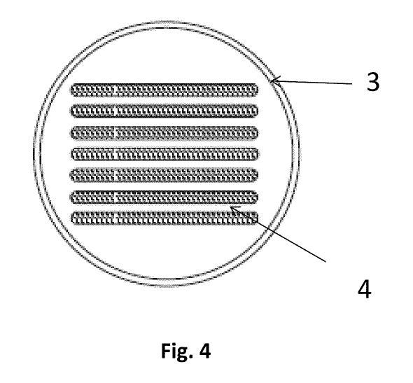

Now referring to FIG. 1, there is shown the chew composition according to present invention rolled into a mass 1. Further as shown in FIG. 2, the rolled mass 1 could be added to a paper or cloth wrap 2 which can be then rolled and sealed from all sides to provide rolled wrappers that could be placed between cheek and gums for slowly releasing the active ingredients into the oral cavity. The paper or cloth wrappers are permeable to liquid including saliva wherein the paper or cloth wrap 2 do not dissolve or our damaged due to liquid. More specifically, the paper or cloth wrap 2 permeability permits the saliva to diffuse inside the wrap or pouch and wet the ingredients and further allows the active ingredients from the pouch or wrap to diffuse out into the user's oral cavity. The rolled wrappers could be stored in a container 3 as shown in FIG. 3, the container 3 is cylindrical with a cap on top. FIG. 4 shows the rolled wraps 4 placed in the container 3. It is obvious however that the shape and design of container 3 could be easily varied and the container 3 could further be provided with ornamental valve.

The wrapper or pouch could be produced from materials such as paper, fabric, non-disposable polymers, and other suitable materials, provided the material is food grade. Such materials are commonly used in tea bags, tobacco chew pouches etc. It is obvious that the wrapper or pouch could be produced in different volumes for example the pouch could be produced having a length of 30 mm, breadth 15 mm and 10 mm thick. Further provided, the size of pouch should not be large that it is difficult to accommodate the pouch between the cheek and the gums.

In one embodiment, the composition includes 45-85% by weight of pulverized green tea leaves, 2-5% by weight of baking powder, 2-5% by weight of turmeric powder; 5% by weight of flavoring agent, and further may include humectant and other ingredients. The composition could be produced by mixing the predetermined quantity of all the ingredients. It is preferably to mix the solid ingredients such as green tea leaves and turmeric powder and thereafter slowly adding the semi-solid and liquid ingredients with thorough mixing.

The composition could also be provided loose in containers wherein the user could transfer small amount of composition from the container into a wrap and then fold the wrap multiple times. The folded wrap could be then placed in the mouth, which could be economical than the sealed pouches. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

What is claimed is:

1. An oral composition for providing a person a habitual chewing satisfaction and enamel cleaning, said natural composition comprising:
45-85% by weight of pulverized green tea leaves green tea leaves mixed with 2-5% by weight of turmeric powder mixed with 2-5% by weight of baking powder mixed with 5% by weight of flavoring agent wherein the oral composition is enclosed in a wrap or pouch that is adapted to be place between the cheek and the gums for slowly releasing the ingredients into the oral cavity providing various health benefits to the user such as cleaning of teeth, providing habitual tobacco chewing satisfaction etc.

2. The oral composition according to claim 1, wherein said green tea leaves are produced in grounded form.

3. The oral composition according to claim 1, wherein said green tea leaves are produced in pulverized form.

4. The oral composition according to claim 1, wherein said natural flavoring agent is selected from wintergreen, mint or a combination thereof.

5. An oral pouch enclosing a composition for providing a person a habitual chewing satisfaction and enamel cleaning, said composition comprising:
a. green tea leaves;
b. turmeric powder;
c. baking powder; and
d. at least one natural flavoring agent;
said pouch is adapted to be placed between the cheek and gum wherein said composition is wetted by saliva and the composition is slowly released in an oral cavity of the person.

6. The oral pouch according to claim 6, wherein said pouch is made of a paper material.

7. The oral pouch according to claim 6, wherein said pouch is made of a cloth material.

8. The oral pouch according to claim 6, wherein said green tea leaves are produced in grounded form.

9. The oral pouch according to claim 6, wherein said green tea leaves are produced in pulverized form.

10. The oral pouch according to claim 6, wherein said natural flavoring agent is selected from wintergreen, mint or a combination thereof.

11. The oral composition of according to claim 6, wherein said composition comprises 45-85% by weight of pulverized green tea leaves, 2-5% by weight of baking powder, 2-5% by weight of turmeric powder and 5% by weight of flavoring agent.
